(19) United States
(12) Patent Application Publication
(10) Pub. No.: US 2024/0335313 A1
(43) Pub. Date: Oct. 10, 2024

(54) ANKLE FOOT ORTHOSIS AND METHOD OF MAKING AND USING

(71) Applicant: Step Ahead Medical LLC, Medford, NJ (US)

(72) Inventors: Rick Raimondo, Medford, NJ (US); John Dudley, West Chester, PA (US); Nadia Din, Barnegat, NJ (US); Charles Soltner, Mullica-Hill, NJ (US)

(21) Appl. No.: 18/749,768

(22) Filed: Jun. 21, 2024

Related U.S. Application Data
(63) Continuation of application No. PCT/US2022/082353, filed on Dec. 23, 2022.
(60) Provisional application No. 63/293,218, filed on Dec. 23, 2021.

(51) Int. Cl. A61F 5/01

(52) U.S. Cl. CPC A61F 5/0111

(57) ABSTRACT

An ankle foot orthosis (“AFO”) is provided. The AFO includes an upright portion that can be attached to the user's calf. It also includes a footbed with symmetrical lateral and medial sides such that the AFO may be worn on the user's right or left foot. Further, a non-slip coating may be applied to the underside of the footbed, such that the AFO may be worn without a shoe. Additionally, the AFO may be reversibly attached to a walking boot to keep it clean and dry. A removable liner may be attached to the AFO for sanitary and comfort purposes.

### RELATED APPLICATIONS

This application claims priority from application PCT/US2022/082353, filed Dec. 23, 2022, and provisional application 63/293,218 filed Dec. 23, 2021, the entire disclosures of which are incorporated herein by reference.

### TECHNICAL FIELD

This disclosure generally relates to an ankle foot orthosis (“AFO”) and method for bracing a patient's lower leg, ankle and foot throughout the period of recovery from an injury or surgery.

### BACKGROUND

Certain types of injuries and surgeries require the patient's ankle, foot and lower leg to be immobilized and supported to facilitate recovery. In such cases, an AFO can be used to stabilize and support the foot and ankle in an optimal position. AFOs are sized according to foot length and calf circumference and they are made to fit either the right or left foot. Understandably, there are many foot and calf size combinations that need to be stocked, for both the right and left foot, in order to ensure that the provider has the correct AFO on hand for any given patient. This creates storage and inventory cost problems for providers.

Further, many of the AFOs on the market have to be custom fit to the user. This often requires a lengthy process that includes sending a patient to an outside lab for fabrication of the AFO. The process is expensive, time consuming and frequently delays treatment for several weeks or more.

Another problem with existing AFOs is that the soles are often hard plastic and they slide on the floor when worn without a shoe. This may cause compliance issues as patients may remove the AFO, rather than risking a fall, if they are indoors or get up from resting. Noncompliance or removal of the bracing system prematurely may result in harm to the foot, ankle and leg. For instance, displacement of a fracture or delayed healing of a wound, tendon or ligament will prolong the period of recovery and rehabilitation. Leaving the foot and ankle unprotected and not maintaining them at the proper angle, even intermittently, can lead to contractures or other injuries that may require more healing time, additional surgery and more extensive rehabilitation than if the ankle were braced in the correct position at all times. This is a prevalent problem among patients.

Moreover, a patient's needs change over the course of recovery, but currently available AFOs are not designed to easily adapt. Currently available AFO's are not designed to work with a walking boot or for use without a shoe. With this in mind, it would be advantageous if the AFO could be worn in conjunction with a walking boot to provide further support and keep the AFO clean. A dirty boot from outside exposure could be removed while indoors and the AFO would be left to protect the ankle and foot. As recovery progresses, the same AFO that worked with the walking boot or protective shell could be used alone or in conjunction with a shoe.

What is needed is an AFO that fits either foot for a wide range of patients without the delay and expense of custom fitting and that can meet most or all of the patient's needs throughout the stages of recovery and throughout the patient's normal daily routine.

### SUMMARY

The present disclosure provides an AFO that can fit a variety of leg and foot sizes and either the right or left foot.

The AFO can be configured to maintain a patient's foot, ankle and lower leg at the prescribed angle to allow healing and avoid, for example, contracture. The AFO is formed from a rigid or semi-rigid material, e.g., plastic. The AFO has a footbed that attaches to and supports the patient's foot and an upright portion(s) that attaches to the calf and forms the proper angle with the footbed. The angle between the footbed and the upright portion may be generally at or near perpendicular. The upright portion attaches to the patient's leg and foot with, e.g., straps.

The AFO can be used with a walking boot. In some non-limiting embodiments, the AFO may be removably attached to a walking boot. Advantageously, this allows patients to remove, e.g., a dirty walking boot rather than bringing it into bed with them and still have enough support from the AFO to maintain the proper position of the foot, ankle and lower leg. The AFO may also be worn by itself when less support is required. The AFO alone may be preferable for sleeping or resting during the day. During later stages of recovery, it can be worn within a shoe.

The AFO may be reversibly attached to a walking boot via straps that pass though the base of the walking boot or, in another example, a strap may connect the AFO to a fabric liner that is attached to the walking boot. The AFO may also be attached to the walking boot using a variety of mechanisms that include but are not limited to, e.g., other strap configurations, a cable, hooks, latches, hook and loop, reversible adhesives, a track and rail system, a knob and rail connection, a continuous strap, a magnetic device, a locking/unlocking mechanism, a hinge mechanism, a cantilever mechanism, and/or a snap fit connection.

The underside of the AFO may have a thin, slip-resistant coating. This reduces the risk of falling if the AFO is being worn by itself, such as at night.

Further, the AFO can be made very thin, such that it can fit into a shoe. The shoe may be a custom shoe, a protective shell, or an off-the-shelf shoe suitable for daily wear.

Finally, a padded insole may optionally be placed in the footbed of the AFO. The insole may provide cushioning and/or arch support.

In a first embodiment, this disclosure provides an AFO having an upright portion configured to attach to the user's calf and a footbed that is symmetrical about its midline such that the AFO may be worn on the user's right or left foot. The AFO further has a non-slip coating on the footbed so that the AFO may be worn without a shoe.

In one aspect of the embodiments described herein, the AFO may be configured to fit inside a shoe. The shoe may be a custom shoe or protective shell. Preferably, the shoe can be an off-the-shelf shoe.

In a further aspect, the AFO is less than 1.5 cm thick. More preferably, the AFO is less than 0.5 cm thick.

In another aspect of the embodiments described herein, the AFO may be configured to provide adequate protection without a shoe. In such embodiments, the AFO may be at least 0.75 cm thick, and preferably at least 1.5 cm thick. Alternatively, in such embodiments, the AFO may be at least 2 cm thick.

In a further aspect of the instant disclosure, the AFO may include one or more sidewalls on the lateral and/or medial sides of the footbed. Such sidewalls may provide additional protection if, for example, the user is wearing the AFO without a walking boot or shoe. The sidewalls may also provide an attachment for a boot liner, e.g., the sidewall may include a slot, straps, buckles, snaps, buttons, hook and loop, etc.

In another aspect, the AFO may be configured to attach to a walking boot (also referred to herein as a “protective cover” or “protective shell”). The AFO may be attached to the walking boot by a rail, a track, a rail and a protrusion, a flush mount bracket, a snap fit connection, a hook and loop connection, a magnet, a strap and a continuous strap, a hinge mechanism, a cantilever mechanism, cable(s), hooks, latches, reversible adhesives, a locking and unlocking mechanism, or by other suitable means.

In a further aspect, the upright portion and the footbed of the AFO can be formed as a single unit. In the alternative, the upright portion and the footbed may be formed separately and connected together. Advantageously, this may allow different sized upright portions to be connected to a footbed. The upright and footbed portions may also be made of heat moldable material to allow for easy adjustments. In another alternative, the upright portions may be two upright posts, preferably positioned on the lateral and medial sides of the patient's calf.

In an aspect of the first embodiment, the AFO can be configured for insertion into a shoe. For example, at a later stage of recovery the walking boot may not be required, but some bracing may still be needed for stability and to maintain the proper angle of the foot. At such a stage of recovery, the AFO may be removed from the boot and used with a shoe or it may be used on its own without a shoe.

In a second embodiment, a method of making and using the AFO is disclosed.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 is a front perspective view of an AFO according to this disclosure.

FIG. 2 is a side view of the AFO shown in FIG. 1.

FIG. 3 is a side view of an AFO according to this disclosure.

FIG. 4 shows a front perspective view of the AFO depicted in FIG. 1 and also including optional bands.

FIG. 5 shows a perspective view of the AFO depicted in FIG. 4 including an optional attachment mechanism.

FIG. 6 shows a rear perspective view of the AFO depicted in FIG. 1 including an optional attachment mechanism.

FIG. 7 shows a front perspective view of an AFO according to this disclosure.

FIG. 8 shows a rear perspective view of an AFO depicted in FIG. 7.

FIG. 9 shows a side perspective view of an AFO depicted in FIG. 7.

FIG. 10 shows a cross-sectional view of an AFO depicted in FIG. 7 and also including an optional walking boot.

FIG. 11 shows a cross-sectional view of the AFO depicted in FIG. 7 and also including an optional walking boot.

FIG. 12 shows a rear perspective view of the AFO depicted in FIG. 7 and also including an optional walking boot.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

In this disclosure, terms such as “horizontal” and “vertical” are generally used to establish positions of individual components relative to one another rather than an absolute angular position in space. Further, regardless of the reference frame, in this disclosure terms such as “vertical,” “parallel,” “horizontal,” “right angle,” “rectangular” and the like are not used to connote exact mathematical orientations or geometries, unless explicitly stated, but are instead used as terms of approximation. With this understanding, the term “vertical,” for example, certainly includes a structure that is positioned exactly 90 degrees from horizontal, but should generally be understood as meaning generally positioned up and down rather than side to side. Other terms used herein to connote orientation, position or shape should be similarly interpreted. Further, it should be understood that various structural terms used throughout this disclosure and claims should not receive a singular interpretation unless it is made explicit herein.

Further, it should be understood that all terms used throughout this disclosure and claims, regardless of whether said terms are preceded by the phrases “one or more, “at least one, or the like, should not receive a singular interpretation unless it is made explicit herein. That is, all terms used in this disclosure and claims should generally be interpreted to mean “one or more” or “at least one.”

The AFO 2 maintains the foot and ankle at a prescribed angle. For example, when the foot and ankle are suspended to prevent weight bearing, the AFO can prevent or minimize the natural plantar flexion that occurs when the foot is unweighted or hanging free. The AFO can also prevent or minimize the degree of dorsiflexion when the user walks on the foot.

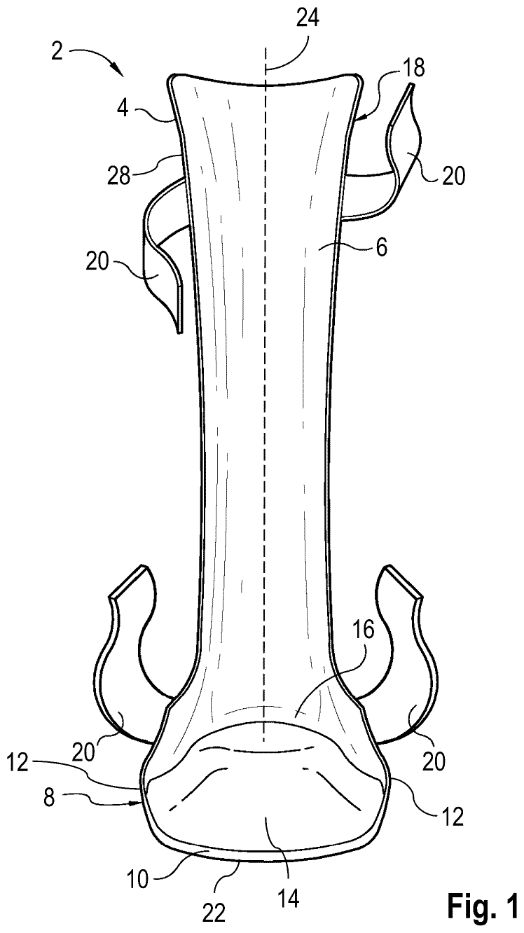

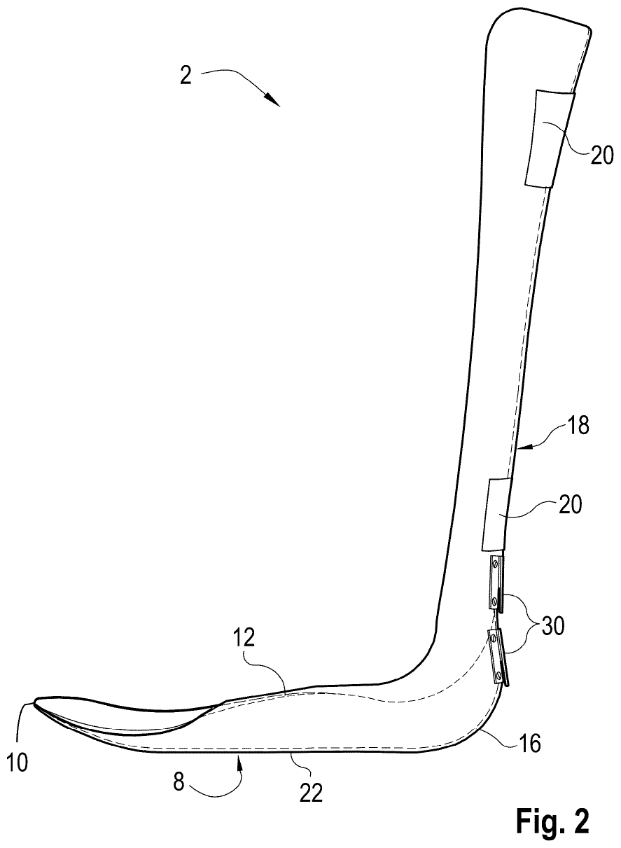

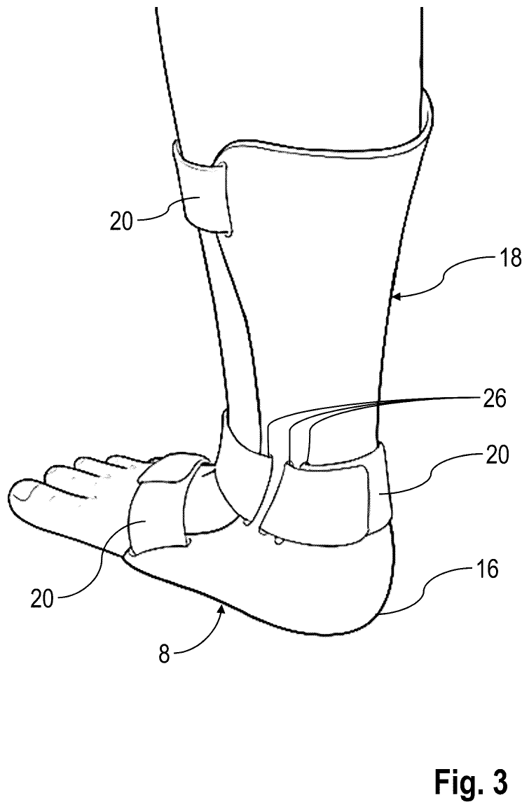

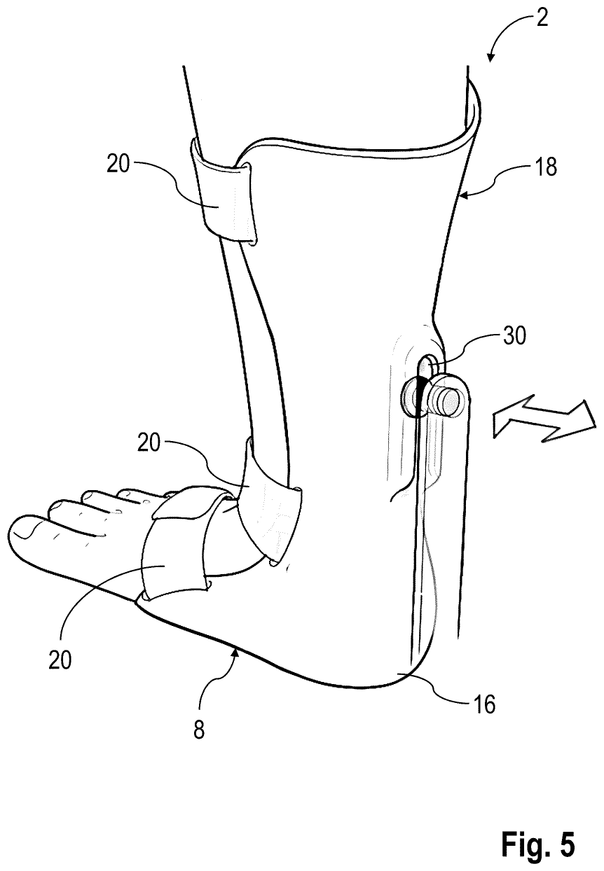

As shown in FIG. 1, the AFO 2 includes a footbed 8, which extends from the toe area 10 to the heel 16. The footbed 8 may angle upward slightly at the toe area 10, which can be seen in FIG. 2. In the alternative, the footbed 8 may be generally planar. In another alternative, not shown, the bottom surface of footbed 8 may be curved to facilitate walking. In another alternative, as shown in FIGS. 3 and 5, the footbed may be made shorter than the foot, ending at the metatarsal heads (balls of the feet) for comfort and better fit when the AFO is worn in regular shoes.

A sidewall 12 extends upward from the footbed 8 between the heel 16 and the toe area 10 to protect the lateral and/or medial sides of the foot. Preferably, there is a sidewall 12 on both the lateral and medial sides of the footbed 8.

The heel 16 connects footbed 8 to upright portion 18. Heel 16 is, preferably, generally semi-spherical or semi-ovoid to accommodate the patient's heel.

The upright portion 18 of AFO 2 extends upward from the heel 16. The upright portion 18 is curved to fit around the posterior portion of the lower leg. In the embodiments shown herein, the anterior side of the leg is exposed. However, in alternative embodiments, the upright portion may surround the lower leg to a greater or lesser extent.

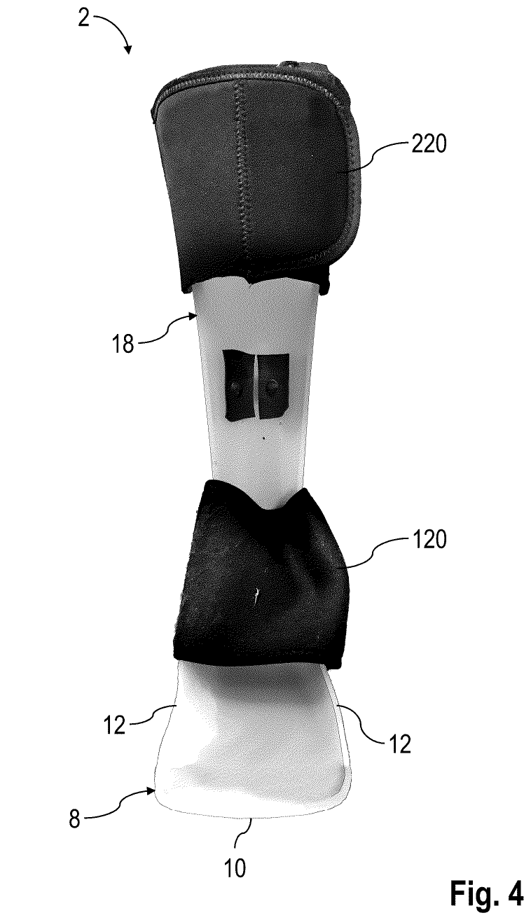

As can be seen in FIG. 3, the AFO 2 may be secured to the patient's leg and foot using straps 20. The straps 20 may, by way of non-limiting example, be fastened with hook and loop, snaps, buttons, an elastomeric material, or any suitable material known in the art. Straps 20 may pass through slots 26 to ensure that they remain in a fixed position and do not slip or twist during use. The AFO 2 may also be secured to the foot and/or leg by some other device such as a bandage, a wrap, a sock or some combination of methods. For example, FIG. 4 shows two additional alternative bands 120, 220 that may be used to secure AFO 2 to the user's foot and leg. Bands 120, 220 may be used in conjunction with slots 26. Various straps 20 and bands 120, 220 may be used interchangeably and in combination.

The footbed 8, upright portion 18 and heel 16 may be formed as a single, monolithic piece, such as by casting or a similar technique. In the alternative, the footbed 8 and upright portion 18 may be formed in separate pieces that may be securely fastened together using, for example, latches, slots and tabs, welding, screws, or any means known in the art.

The exterior shell 4 of AFO 2 may be formed from a material that provides structure for stabilizing the foot and ankle, such as rubbers, plastics, silicones, carbon fibers, lightweight metals, resins, thermoplastic filaments, thermoplastic elastomers, polyurethane elastomers, copolyesters, and combinations or composites thereof. More particularly, the AFO may be formed from polylactic acid, polyvinyl chloride, polyethylene, polypropylene, copolymer, and/or acrylonitrile styrene (ABS).

The interior 6 of the AFO 2 can be made of a softer material for comfort such as fabric or foam, for example, the product sold under the trademark PLASTAZOTE®, manufactured by Zotefoams.

Further, the sole 22 can be coated with an elastomeric material to allow greater traction if the AFO is worn without a shoe or walking boot. For example, the sole 22 may be coated with rubber, polyurethane and/or PVC compounds to minimize the risk of slipping. However, the coating on sole 22 should be thin such that AFO 2 may still be worn inside an off-the-shelf shoe. Preferably, the coating should be less than 1 cm. More preferably, the coating should be less than 5 mm. Most preferably, the coating should be less than 3 mm.

The AFO 2 may be a generic or universal fit to accommodate either the left or right foot as well as a variety of foot sizes and shapes. As shown in FIG. 1, the AFO can be made symmetrical about a centerline 24. As can be seen, the footbed 8 is flat and does not include an arch support, or wider, angled toe space. This allows AFO2 to be used comfortably on either foot. Further, footbed 8 may be made longer to accommodate the full length of a user's foot, as shown in FIG. 2, where the toe area 10 is angled up. In the alternative, it may be made shorter than the length of a user's foot, as shown in FIG. 4, such that the precise shoe size of the user does not matter in fitting him or her with an AFO 2.

In addition to symmetry about the centerline 24, the upright portion 18 may also be formed in various widths to accommodate different calf sizes. Alternatively or in addition, upright portion 18 may be thinner at the top, such that the upper portion 28 (shown in FIG. 1) can be sufficiently deformed to fit a smaller user by pulling the straps 20 together and securing them around the user's calf.

The AFO 2 may be reversibly attached to a walking boot (not shown) using various attachment mechanisms 30. The attachment between the AFO 2 and the walking boot advantageously prevents movement of the AFO 2 when used in conjunction with a walking boot. This improves stability and reduces the risk of friction wounds. Further, particularly when the patient is non-weight bearing, the AFO 2 can maintain the patient's ankle and foot in a prescribed position to avoid, for example, contracture.

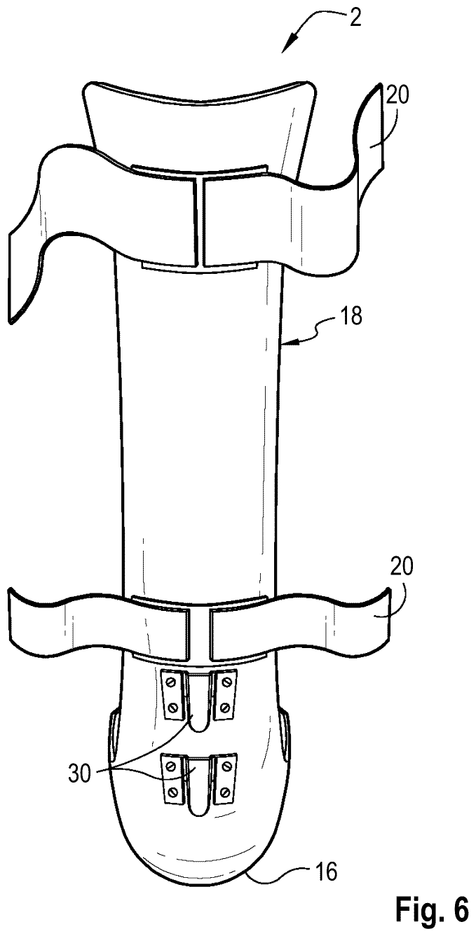

In one non-limiting embodiment, the attachment mechanism 30 may be a flush mount bracket with a clip on the back of the AFO 2, shown in FIGS. 2 and 6, that is configured to connect with a clip on the walking boot. Alternatively, as shown in FIG. 5, the AFO 2 may have a cut out track in the exterior of the upright portion 18 that may cooperate with a knob or post in the walking boot to form the detachable connection. The attachment mechanism 30 may also be any suitable structure known in the art including, e.g., a snap fit connection, straps, hook and loop material (e.g., VELCRO®) or the like. Other embodiments are also contemplated. For example, the AFO 2 may be attached to the footbed of a walking boot by various means such as straps, hook and loop material, hooks, latches, reversible adhesives, hinges, cantilever devices, cables, locking and unlocking mechanisms, magnets, snap fit connections or any other suitable means.

The AFO 2 may also be used with a shoe. If a patient has recovered to the extent that a walking boot or other assistive device is no longer required, the AFO 2 can continue to provide stability and positioning for the foot and ankle, but without the extra structure of, for example, a full walking boot.

The AFO 2 may also be worn by itself, for example, when users are sleeping, sitting down or resting. The AFO 2 will continue to provide stability and proper positioning of the foot, just with a lower degree of protection from external forces, which is appropriate for activities such as sleeping or relaxing.

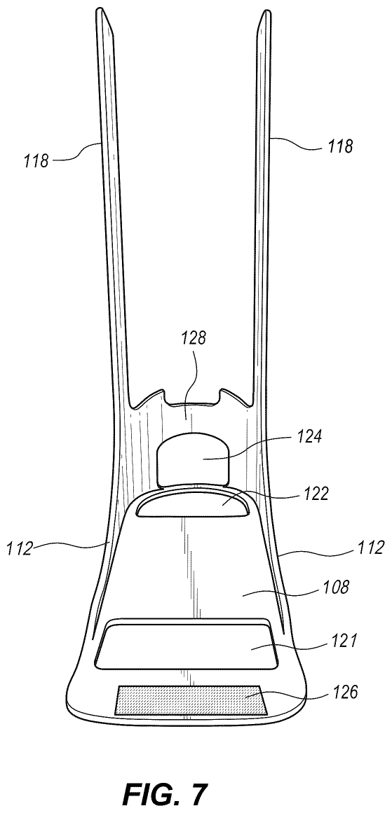

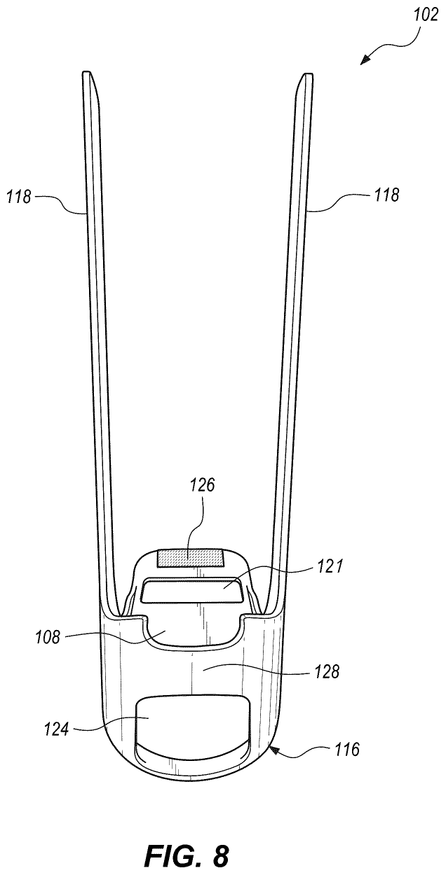

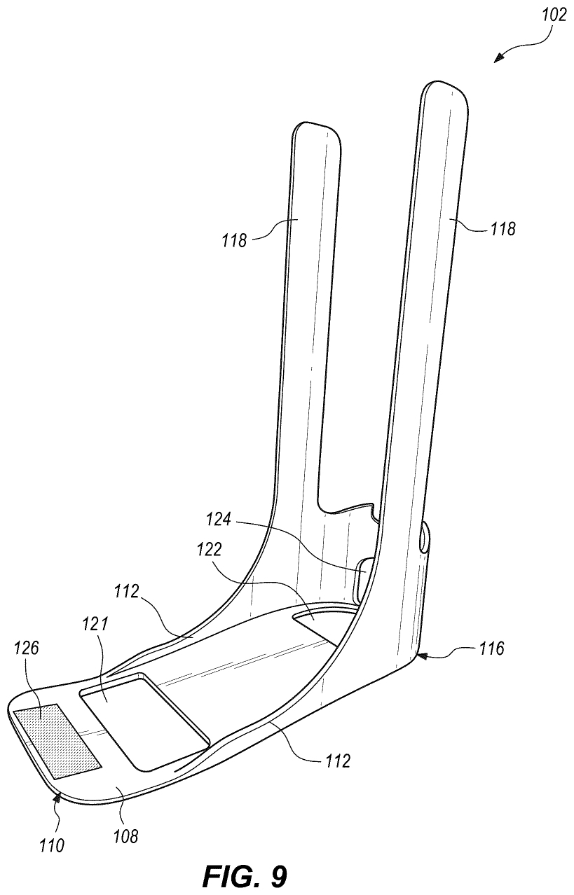

An alternative embodiment of an AFO according to this disclosure is shown in FIGS. 7-9. The AFO 102 includes a footbed 108, which extends from the toe area 110 to the heel 116. The footbed 108 may angle upward slightly at the toe area 110, which can be seen in FIG. 9. In the alternative, the footbed 108 may be generally planar.

A sidewall 112 may extend upward from the footbed 108 between the heel 116 and the toe area 110 to protect the lateral and/or medial sides of the foot. Preferably, there is a sidewall 112 on both the lateral and medial sides of the footbed 108. In the alternative, AFO 102 may be made without a sidewall 112, e.g., to make it easier to fit within a shoe.

The heel 116 connects footbed 108 to upright portions 118. Heel 116 may be generally semi-spherical or semi-ovoid to accommodate the patient's heel. In the alternative, there may be a cutout 124 to accommodate the patient's heel.

The upright portions 118 of AFO 102 extend upward from the heel 116. The upright portions 118 extend up the lateral and medial sides of the lower leg such that the anterior and posterior sides of the lower leg are exposed. A boot liner (not shown) may be removably attached to upright portions 118 in some embodiments. The boot liner may be further be removably attached to AFO 2 at, e.g., an attachment region 126, with, e.g., a hook and loop connection.

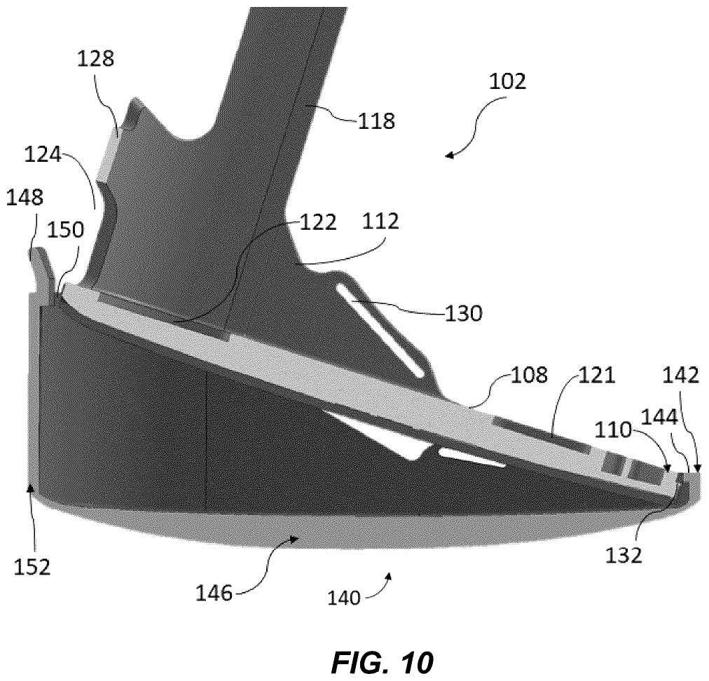

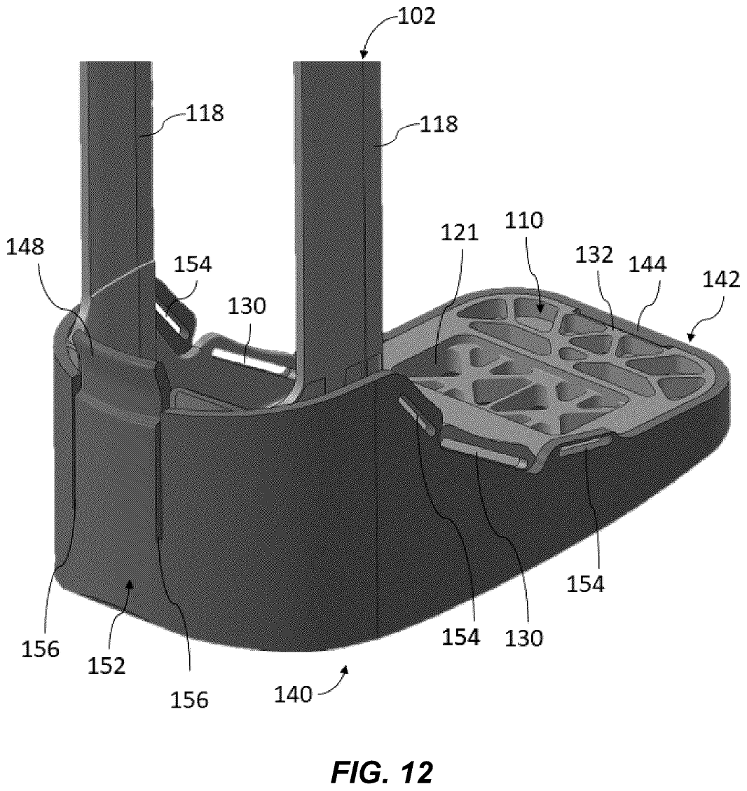

Upright portions 118 may be connected by cross piece 128. Cross piece 128 may extend behind the lower leg and ankle area of the patient and may provide additional structural support and rigidity to uprights 118. Further, as described below in connection with FIGS. 10-12, cross piece 128 provides ample room for attachment to a walking boot or protective cover.

AFO 102 may optionally include cutouts 121 and/or 122 to offload weight from the patient's metatarsals and/or heel, respectively. The cutouts 121 and 122 may extend fully through the thickness of footbed 108 as shown in FIGS. 7-9. Alternatively, cutouts 121 and 122 may extend only partially through the thickness of footbed 108, as shown in FIGS. 10-12. In an additional option, padding (not shown) may be placed inside cutouts 121 and 122 for additional comfort and support.

AFO 102 may optionally be removably attached to a walking boot 140 as shown in FIGS. 10-12. Toe area 110 of AFO 102 may have a notch 132 configured to cooperate with ridge 144 in the toc area 142 of walking boot 140 such that the AFO 102 may be attached to walking boot 140.

Further, the heel 152 of walking boot 140 may have a latch 148 with a ridge 150. The latch 148 may cooperate with cross piece 128 of AFO 102 to attach AFO 102 to walking boot 140. The latch 148 may comprise two slots 156 cut into heel 152 of walking boot 140 such that latch 148 can be pulled back to release AFO 102 from walking boot 140.

Additionally or alternatively, there may be slots 130 in the sidewalls 112 of AFO 102. The slots 130 may accommodate a strap (not shown) that secures AFO 102 to the patient's foot. It is also contemplated that there may be buckles or posts (not shown) connected to sidewalls 112 that may be used to secure AFO 102 to the patient's foot. Further, there may be slots 154 in walking boot 140 that may accommodate a strap (not shown) that secures walking boot 140 to the patient's foot.

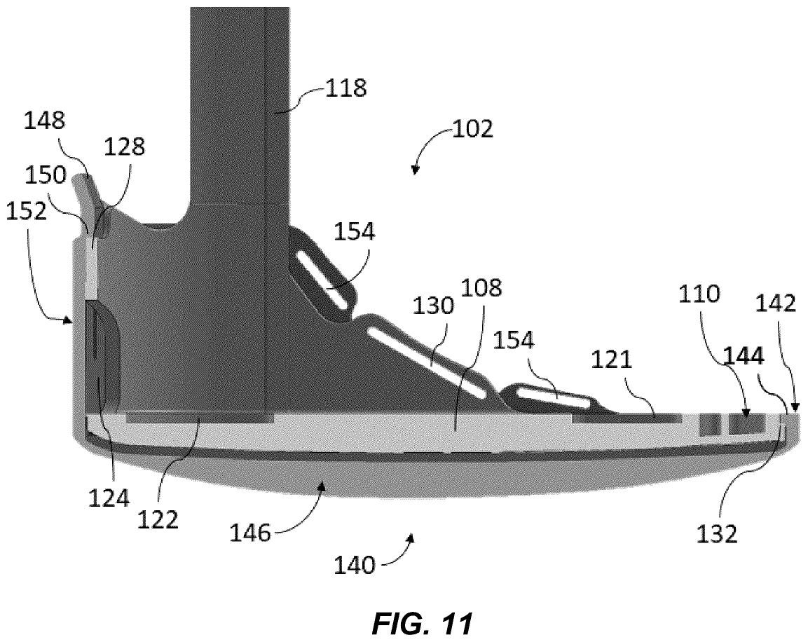

The sole 146 of walking boot 140 may, optionally, be curved (or rocker shaped), as shown in FIGS. 10 and 11. In the alternative, the sole 146 of walking boot 140 may be flat.

### Method of Making and Using the AFQ

The upright portion 18, heel 16 and footbed 8 of AFO 2 may be formed as a single piece by, for example, casting in a mold, extrusion, 3-D printing, machining or other suitable means. An attachment mechanism 30 may be incorporated either when the AFO 2 is formed or after the AFO is formed. Straps 20 or bands 120, 220 may then be added to the AFO 2. The underside of footbed 8 may then be partially or fully coated with a slip-resistant coating.

The AFO 2 is typically symmetrical about the centerline 24, such that it can be worn on either the right or left foot. An insole may optionally be added to the AFO for arch support. The insole may be a generic or off-the-shelf insole, or it may be a custom insole.

The AFO 2 is attached to the injured foot or ankle by placing the user's foot onto the footbed 8 and the user's leg into the upright portion 18. Straps 20 may then be fastened to secure the AFO 2 to the user's foot and leg. The wider bands 120, 220 shown in FIG. 4 may, alternatively, be used to secure the AFO 2 to the patient's foot and leg.

The AFO 2 can then be attached to a walking boot if needed. The user's leg may be placed into the walking boot and the AFO 2 may then be secured using an attachment mechanism 30, or by any other attachment mechanism described elsewhere herein. Notably, in a case where the user cannot bear weight on the injured foot, an AFO 2 can maintain the foot at the prescribed angle, rather than letting it dangle from the ankle.

Alternatively, the user's foot may be placed in an ordinary shoe. In another alternative, if the user is resting or otherwise remaining sedentary, the AFO 2 may be worn without a walking boot or shoe.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. An ankle foot orthosis (“AFO”), comprising:
an upright portion configured to attach to a user's calf;
a footbed comprising a lateral side and a medial side and configured to attach to the user's foot, wherein the lateral side and the medial side of the footbed are symmetrical, whereby the AFO is configured to be worn on the user's right or left foot; and
a non-slip coating disposed on the footbed, whereby the AFO provides traction when worn without a shoe.

2. The AFO of claim 1, wherein the AFO is configured for insertion into a shoe.

3. The AFO of claim 1, wherein the AFO is configured for use without a shoe.

4. The AFO of claim 1, wherein the AFO comprises a first cutout in the heel and a second cutout in the footbed.

5. The AFO of claim 4, wherein padding is inserted in the first and/or second cutout.

6. The AFO of claim 1, further comprising a sidewall on the lateral and medial sides of the footbed.

7. The AFO of claim 1, wherein the AFO is configured to attach to a walking boot.

8. The AFO of claim 7, wherein the AFO is attached to the walking boot by one of a rail, a track, a rail and a protrusion, a flush mount bracket, a snap fit connection, a hook and loop connection, hooks, latches, reversible adhesive, cables, locking and unlocking mechanisms, a magnet, a hinge connection, a cantilevered connection, a strap and a continuous strap.

9. The AFO of claim 7, wherein the AFO is configured to reversibly attach to the toe area of a walking boot.

10. The AFO of claim 7, wherein the AFO is configured to reversibly attach to the heel area of a walking boot.

11. The AFO of claim 7, wherein the walking boot is a protective shell or a custom shoe.

12. The AFO of claim 1, wherein the upright portion, the heel and the footbed are integrally formed.

13. The AFO of claim 1, wherein the upright portion comprises a medial side post and a lateral side post.

14. The AFO of claim 1, configured to attach a fabric liner.

15. The AFO of claim 1, wherein the bottom surface of the footbed is curved.
